(19) United States
(12) Patent Application Publication
(10) Pub. No.: US 2009/0247833 A1
(43) Pub. Date: Oct. 1, 2009

(54) ADAPTER FOR REMOVABLY COUPLING A CAMERA TO A LARYNGOSCOPE AND LARYNGOSCOPE AND SYSTEM USING SAME

(76) Inventor: David T. Tanaka, Durham, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/416,840

(22) Filed: Apr. 1, 2009

Related U.S. Application Data
(60) Provisional application No. 61/041,394, filed on Apr. 1, 2008.

(51) Int. Cl. A61B 1/267; A61B 1/04; G09B 23/28

(52) U.S. Cl. 600/188; 434/262

(57) ABSTRACT

An adapter for removably coupling a camera to a laryngoscope and a laryngoscope and system using same is disclosed. According to one aspect, the subject matter described herein includes an adapter for removably coupling a camera to a laryngoscope, the laryngoscope having a laryngoscope blade with a first end and a second end. The adapter includes an adapter body having a first attachment member for attaching the adapter body to a laryngoscope and a second attachment member for attaching a camera to the adapter body and for holding the camera in a predetermined orientation with respect to the laryngoscope.

### RELATED APPLICATIONS

This application claims the benefit of U.S. Provisional Patent Application Ser. No. 61/041,394, filed Apr. 1, 2008; the disclosure of which is incorporated herein by reference in its entirety.

### TECHNICAL FIELD

The subject matter described herein relates to increasing the proficiency of laryngoscope users. More particular, the subject matter described herein relates to an adapter for removably coupling a camera to a laryngoscope and a laryngoscope and system using same.

### BACKGROUND

Major clinical advances have been made in the area of newborn care over the past 30 years. Yet despite these advances, approaches to training young health care providers in the critical skill of intubation have not changed since the creation of the laryngoscope handle and blade. In this process, the novice incubator is exposed to a number of audio/visual and simulator aids to intubation. Following this introduction, the novice intubator's first human intubation often takes place in a clinical intubation setting. On the first and subsequent attempts, the novice intubator is “coached” by an experienced intubator during the procedure. The significant drawback of this training is that the experienced intubator cannot view what the novice intubator is seeing since there is insufficient space for both to see the view into the larynx at the same time. Not surprisingly, both the number of mis-identifications of oral and pharyngeal structures and intubation failure rates are high.

In a recent survey of Neonatal Program Directors, the vast majority expressed concern that the onset of duty hour restrictions, coupled with the general improvement of medical care to reduce ventilator days have resulted in poorer intubation skills of pediatric residents. These impressions were subsequently confirmed in a recent Q/A interview wherein we demonstrated that up to 10 to 15 different opportunities were needed to acquire the skill of intubation. Since our current residents average only 5 intubation opportunities over their three year Residency period, the concern that they may not be learning this skill appears well founded. Accordingly, a better way to teach the skill in the broadest possible clinical setting is desperately needed.

### SUMMARY

According to one aspect, the subject matter described herein includes an adapter for removably coupling a camera to a laryngoscope. The adapter includes an adapter body having a first attachment member for attaching the adapter body to a laryngoscope having a blade with a first end and a second end, and having a second attachment member for attaching a camera to the adapter body and for holding a camera in a predetermined orientation with respect to the laryngoscope.

According to another aspect, the subject matter described herein includes a laryngoscope. The laryngoscope includes a handle and a blade having a first end coupled to the handle and a second end adapted to be inserted into the throat of a patient. The laryngoscope also includes a camera for imaging anatomical structures being probed by the laryngoscope, and an adapter for removably mounting the camera to the laryngoscope blade and for holding the camera in a predetermined orientation with respect to the laryngoscope blade.

According to yet another aspect, the subject matter described herein includes a system for training laryngoscope users. The system includes a laryngoscope, a camera for imaging anatomical structures being probed by the laryngoscope, an adapter for removably mounting a camera to the laryngoscope and for holding the camera in a predetermined orientation with respect to the laryngoscope, and a monitor for displaying output of the camera.

According to yet another aspect, the subject matter described herein includes a method for training laryngoscope users. The method includes attaching a camera to a laryngoscope, using an adapter for removably attaching a camera to the laryngoscope and for holding the camera in a predetermined predetermined orientation with respect to the laryngoscope. A first person performs a medical procedure using the laryngoscope, the output of the camera being displayed for view by a second person. A second person monitors the medical procedure being performed by the first person, by viewing the output of the camera.

### BRIEF DESCRIPTION OF THE DRAWINGS

Preferred embodiments of the subject matter described herein will now be explained with reference to the accompanying drawings, wherein like reference numerals represent like parts, of which:

FIG. 1 is an illustration of an adapter for removably coupling a camera to a laryngoscope, according to an embodiment of the subject matter described herein;

FIG. 2 illustrates in more detail an adapter for removably coupling a camera to a laryngoscope according to an embodiment of the subject matter described herein;

FIG. 3 is an illustration of an adapter for removably coupling a camera to a laryngoscope according to another embodiment of the subject matter described herein;

FIGS. 4A and 4B are illustrations of an adapter for removably coupling a camera to a laryngoscope, the adapter including means for mechanically engaging a focusing mechanism of the camera, according to an embodiment of the subject matter described herein;

FIG. 5 is an illustration of an adapter for removably coupling a camera to a laryngoscope according to another embodiment of the subject matter described herein;

FIG. 6 is an illustration of a system for training laryngoscope users according to an embodiment of the subject matter described herein;

FIG. 7 is a flowchart illustrating an exemplary process for training laryngoscope users according to an embodiment of the subject matter described herein;

FIGS. 8A and 8B are illustrations of an adapter for removably coupling a wireless camera to a laryngoscope according to another embodiment of the subject matter described herein in perspective and exploded views, respectively; and

FIG. 9 is a detailed view of the adapter illustrated in FIGS. 8A and 8B illustrating a mechanism for adjusting the orientation of the laser beam with respect to the laryngoscope according to another embodiment of the subject matter described herein.

### DETAILED DESCRIPTION

In accordance with the subject matter disclosed herein, an adapter for removably coupling a camera to a laryngoscope is provided. Using an adapter for removably coupling a camera to a laryngoscope blade obviates the need to purchase specialty laryngoscopes with cameras built in, which tend to be very expensive. Use of the adapter allows standard, non-camera laryngoscopes to be converted into relatively inexpensive camera laryngoscopes, and the removable nature of the adapter allows one adapter and camera to be used by many different people and with laryngoscopes with many different types, or lengths, of blades. The removal nature of the adapter, together with the detachable camera, also permits easy cold sterilization of the adapter for use between patients.

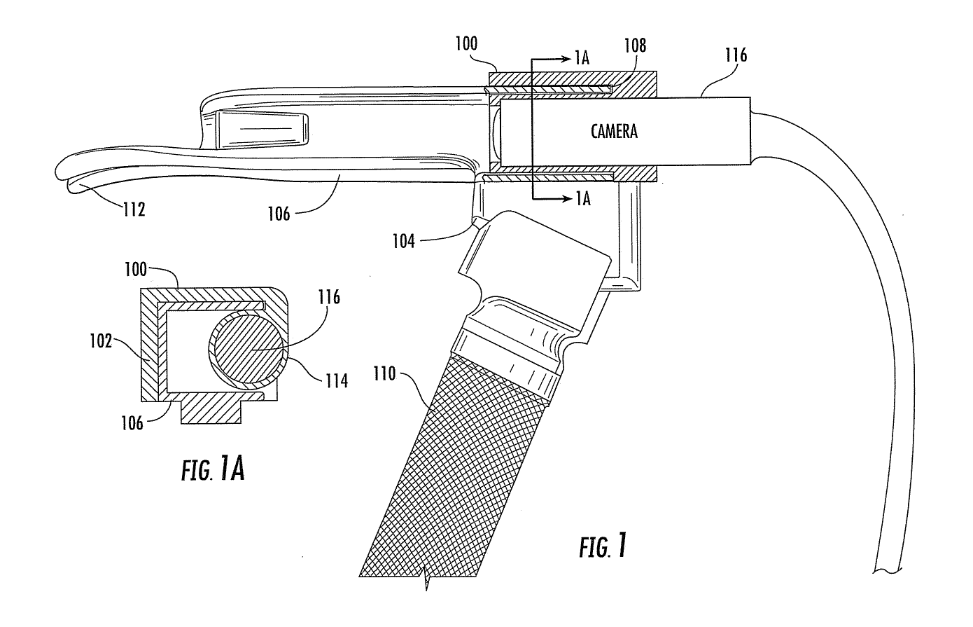

FIG. 1 is an illustration of an adapter for removably coupling a camera to a laryngoscope, according to an embodiment of the subject matter described herein. In FIG. 1, adapter 100 includes an adapter body having a first attachment member 102 for attaching adapter 100 to a laryngoscope 104, the laryngoscope having a laryngoscope blade 106 with a first end 108, which is attached to a laryngoscope handle 110, and a second end 112, which is inserted into the throat of the patient. The adapter body includes a second attachment member 114 for attaching a camera 116 to adapter 100 and for holding camera 116 in a predetermined orientation with respect to laryngoscope 104. For example, adapter 100 may hold camera 116 so as to view objects proximate to the second end of blade 106, e.g., structures of the patient's throat.

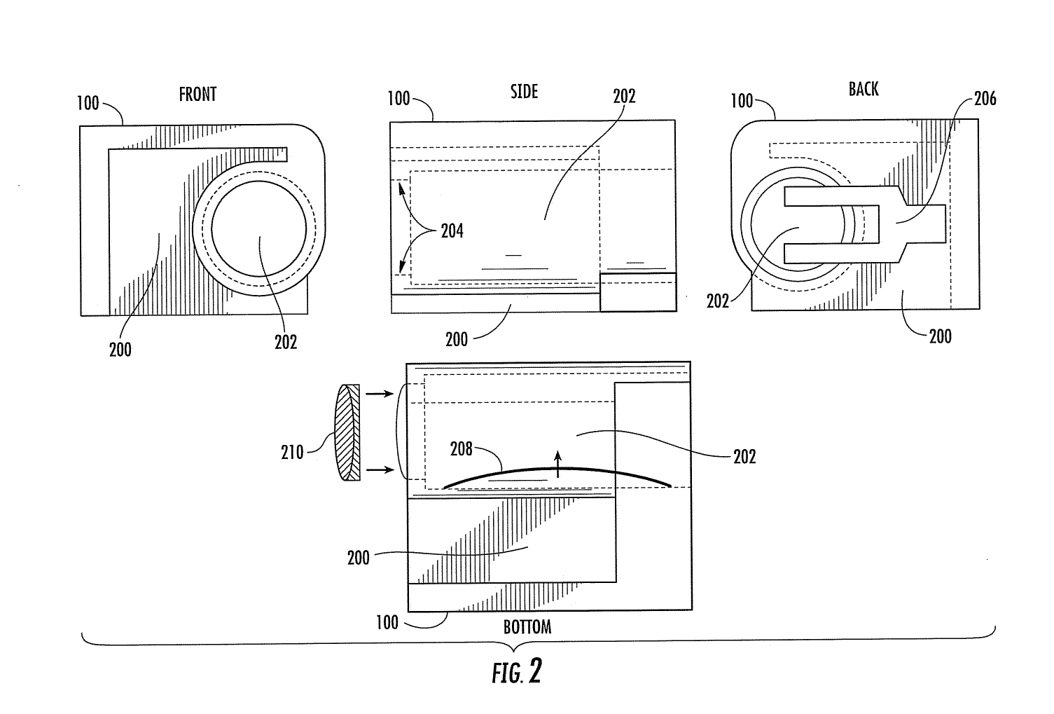

FIG. 2 illustrates in more detail an adapter for removably coupling a camera to a laryngoscope according to an embodiment of the subject matter described herein. FIG. 2 shows front, side, back, and bottom views of adapter 100. In one embodiment, first attachment member 102 comprises a first receptacle 200 disposed within the body of adapter 100 for receiving the first end of laryngoscope blade 106 and for securing the adapter body to the first end of the laryngoscope blade.

In one embodiment, second attachment member 114 may comprise a second receptacle 202 disposed within the body of adapter 100 for receiving camera 116. In one embodiment, second receptacle 202 may be a roughly cylindrical cavity within adapter 100, of dimensions designed to accommodate a cylindrical camera. One end of second receptacle 202 may have an inner lip or constriction 204, so as to prevent camera 116 from sliding forward toward the second end of laryngoscope blade 112 (i.e., toward the patient) or falling out of adapter 100 during handling or use of the laryngoscope.

In one embodiment, a retaining clip 206 may be disposed adjacent to or within second receptacle 202 for securing the camera within second receptacle 202 and thus to the body of adapter 100. For example, retaining clip 206 may be positioned to secure the back end of the camera to prevent the camera from sliding away from the second end of laryngoscope blade 106 (i.e., away from the patient) and out of second receptacle 202 during use of the laryngoscope.

Alternatively, a retaining clip 208 may be disposed along the length of second receptacle 202 and designed to press the body of camera 116 against the inner wall of second receptacle 202 and hold camera 116 in place by friction. For example, a long thin strip of metal or other spring-like material may be inserted between camera 116 and the inner wall of second receptacle 202. By applying a force that attempts to push the camera away from the inner surface of one side of the cylindrical cavity, retaining clip 208 forces camera 116 more strongly against the opposite inner side of the cylindrical cavity, where camera 116 is held in place by friction between the camera body and the inner cavity wall.

In one embodiment, an optically transparent window 210 may be disposed on adapter 100 to permit an unobstructed view of the airway by camera 116 while protecting the lens of camera 116 from contact with and potential contamination by bodily secretions. Window 210 may be a lens for changing the optical properties of the image viewed by the camera, such as providing additional magnification, changing depth of field, providing polarization, filtering, etc.

In an alternative embodiment, second attachment member 114 comprises a clamp for clamping the camera to the adapter body. For example, the camera body may be held between two arms of a clamp structure. In one embodiment, adapter 100 may be constructed of a material that has a slight flexibility, in which case the clamp arms are designed so that inserting camera 116 between the two clamp arms requires that the clamp arms be spread slightly. The force to clamp the camera into place is provided by the clamp arms, which would return to their original, un-spread position but for the presence of the camera body.

In one embodiment, adapter 100 may be designed to fit only one type of blade or only blades of a particular geometry. In an alternative embodiment, adapter 100 may be designed to fit a variety of blade designs. For example, first attachment member 114 may be a receptacle of a shape or cross-section that is designed to fit a variety of blade cross-sections. Similarly, first attachment member 114 may be a clamp that is designed to be attached to a variety of blade designs, or in a variety of locations on the blade.

In one embodiment, adapter 100 may include means for adjusting the orientation of camera 116 with respect to laryngoscope 106. For example, where second attachment member 114 is a clamp structure, camera 116 may be held in a variety of positions by the clamping members, so that the direction in which camera 116 is pointing may be adjusted as needed. In one embodiment, the clamp structure may allow adjustment of the camera orientation relative to blade 106. For example, second attachment member 114 may be a clamp structure that is attached to adapter 100 using a ball and socket joint, which allows the clamp structure and attached camera to swivel and/or rotate with respect to the body of adapter 100. Where second attachment member 114 is a receptacle within the adapter body, shims, sleeves, set screws, or other structures may be used to adjust the orientation of camera 116 within the receptacle.

Similarly, in one embodiment, adapter 100 may include means for adjusting the position, orientation, or location of adapter 100 with respect to laryngoscope 104 or laryngoscope blade 106. For example, adapter 100 may include a clamp structure or other means for attaching adapter 100 to laryngoscope blade 106 at locations other than the first end of the blade, e.g., at various locations along the blade's length.

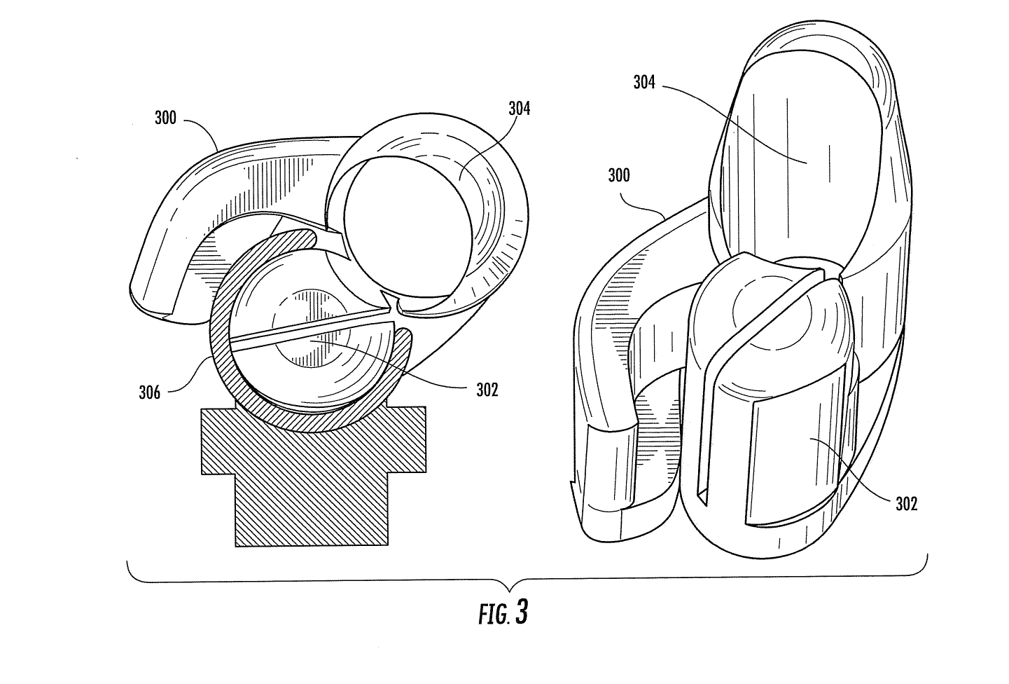

FIG. 3 is an illustration of an adapter for removably coupling a camera to a laryngoscope according to another embodiment of the subject matter described herein. Adapter 300 includes a first attachment member 302 for attaching adapter 300 to a laryngoscope and a second attachment member 304 for attaching a camera to adapter 300. In this embodiment, first attachment member 302 comprises a clamp for clamping the adapter body to the laryngoscope blade. In the embodiment illustrated in FIG. 3, first attachment member 302 is designed to secure adapter 300 to a laryngoscope blade 306, having a curved cross section. First attachment member 302 is designed to fit snugly inside the inner curve of laryngoscope blade 306, securing itself against the blade by applying pressure against opposite positions of the inner wall of the blade. Alternative arrangements include applying pressure against opposite outer walls of the blade, applying pressure against the inner and outer surfaces of the same section of blade, and others. Like the adapter illustrated in FIG. 2, above, in the embodiment illustrated in FIG. 3, second attachment member 304 is a receptacle disposed within adapter 300 for receiving a camera.

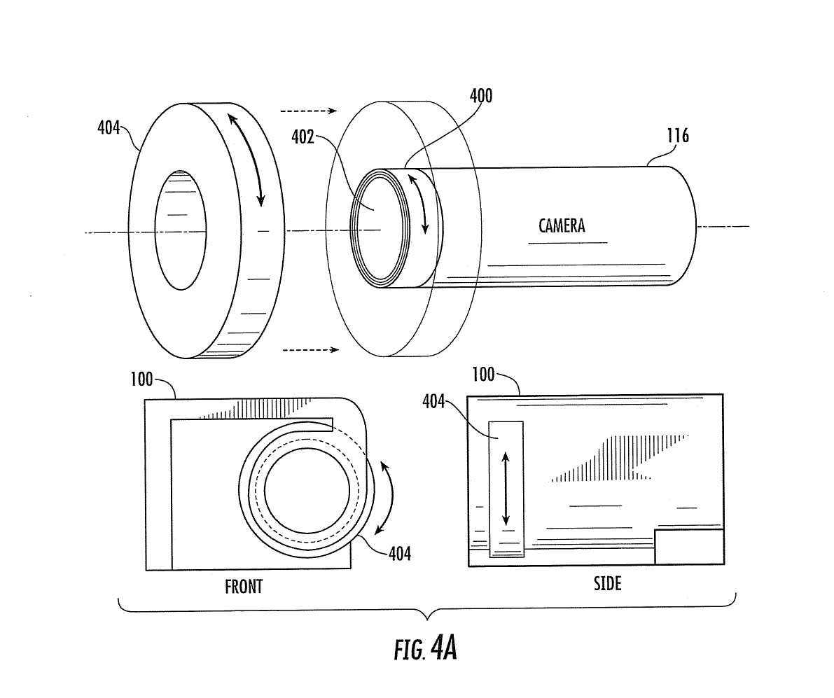

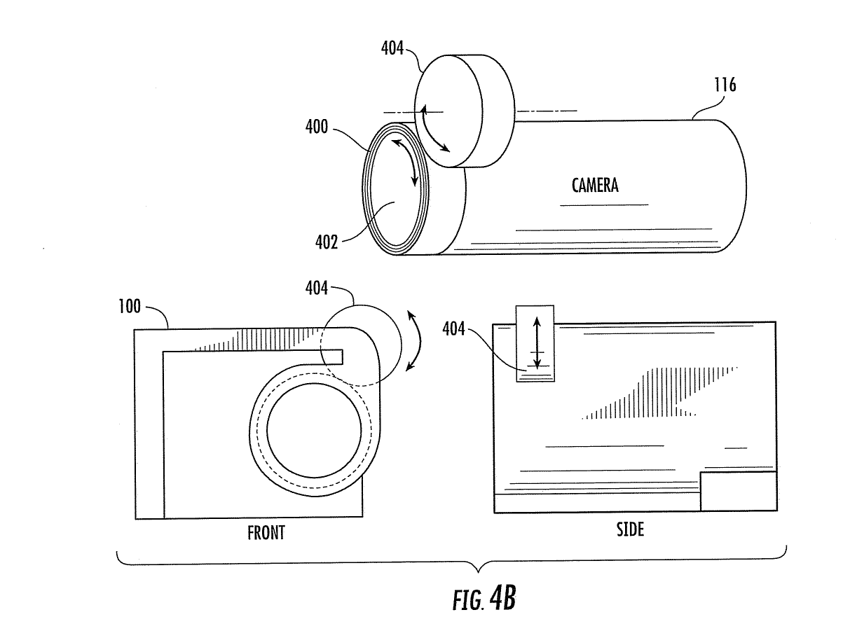

FIG. 4A is an illustration of an adapter for removably coupling a camera to a laryngoscope, the adapter including means for mechanically engaging a focusing mechanism of the camera, according to an embodiment of the subject matter described herein. In one embodiment, camera 180 includes a means for rotating a focusing ring of a lens of the camera, such as focusing ring 400 that, when rotated, adjusts the focal length of lens 402. Adapter 100 includes a focusing knob 404 that is positioned around focusing ring 400 and engaged with focusing ring 400 such that rotating focusing knob 404 also rotates focusing ring 400. In one embodiment, a portion of focusing knob 404 protrudes from the body of adapter 100, positioning focusing knob 404 such that it may be rotated by the user of the laryngoscope. For example, focusing knob 404 may be a knob 404 may be a knurled knob or thumbwheel, positioned so that the user of the laryngoscope may hold the handle of the laryngoscope with the fingers of one hand and adjust the focus of the camera with the thumb of that same hand, thus freeing the other hand to perform other tasks required during the use of the laryngoscope.

FIG. 4B is an illustration of an adapter for removably coupling a camera to a laryngoscope, the adapter including means for mechanically engaging a focusing mechanism of the camera, according to another embodiment of the subject matter described herein. In this embodiment, adapter 100 includes a focusing knob 404 for mechanically engaging with and focusing ring 400, but rather than surrounding focusing ring 400, focusing knob 404 engages focusing ring 400 at one point on the perimeter of focusing ring 400. In this arrangement, rotating focusing knob 404 in one direction will cause focusing ring 400 to rotate in the opposite direction.

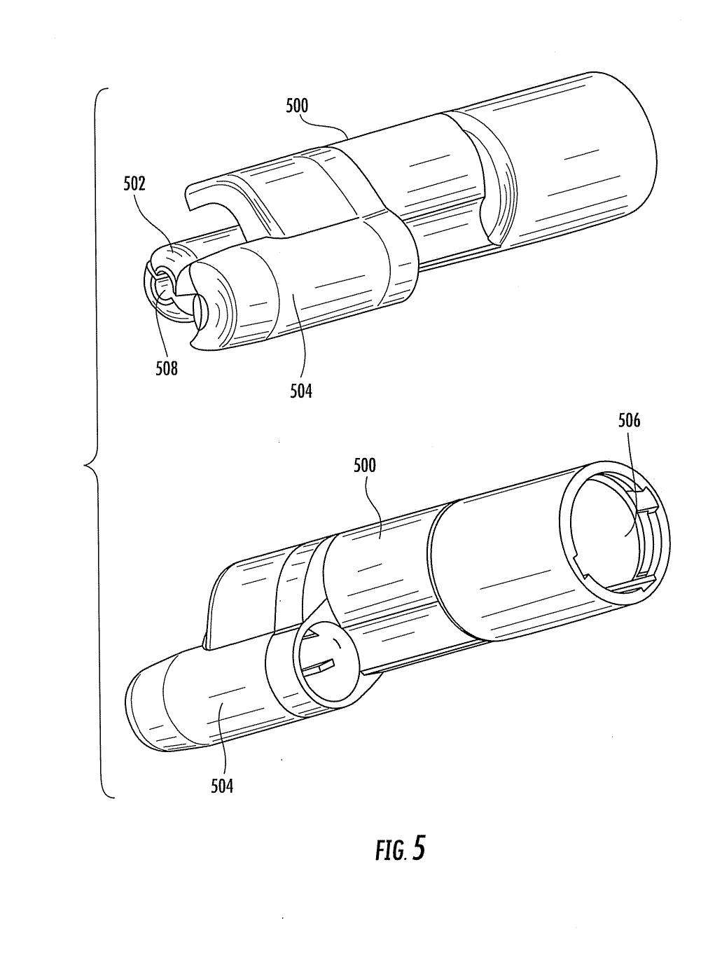

FIG. 5 is an illustration of an adapter for removably coupling a camera to a laryngoscope according to another embodiment of the subject matter described herein. Adapter 500 includes a first attachment member 502 for attaching the body of adapter 500 to a laryngoscope, a second attachment member 504 for attaching a camera to adapter 500, and a third attachment member 506 for attaching a laser to adapter 500 and for holding the laser in a predetermined orientation with respect to the laryngoscope. In the embodiment illustrated in FIG. 5, first attachment member 502 is a clamp structure designed to press outwardly against the inner wall of a laryngoscope blade having essentially C-shaped cross-section. The two arms of first attachment member 502 make contact with portions of the inner wall that are roughly opposite from each other along the circumference of the blade cross-section. In an alternative embodiment, first attachment member 502 may be a first receptacle (not shown) within the body of adapter 500 for receiving one end of a laryngoscope blade.

In one embodiment, second attachment member 504 may be a second receptacle disposed within adapter 500 for receiving camera 116. In one embodiment, third attachment member 506 may be a third receptacle disposed within adapter 500 for receiving a laser pointer or other device for generating a laser beam, herein referred to as the “laser”. In one embodiment, the laser may be a cylindrical member that is insertable into an orifice or aperture 508 such that a beam of laser light is emitted from orifice or aperture 508. Any suitable laser may be used. For example, in one embodiment, the laser may be a Class 1 laser that emits light that is safe to a human user's eyes. The laser is oriented so that the laser beam illuminates structures being probed by the user of the laryngoscope, e.g., structures in the patient's throat. This allows a user of the laryngoscope and a second party who is monitoring the pictures being displayed by camera 116 to unambiguously identify items being viewed by both parties. For example, the user of the laryngoscope may position the laryngoscope so that the red dot of light emitted by the laser is located on a particular structure within the patient's throat, and ask questions about the structure, confirm that the structure is what the user thinks the structure is, and so forth. The third receptacle may be configured to contain a battery powered laser device and also one or more batteries for powering the laser. In an alternative embodiment, third attachment member 506 may comprise a clamp for clamping the laser to the body of adapter 500.

In one embodiment, adapter 500 may include means for adjusting the orientation of camera 116 with respect to the laryngoscope. For example, where second attachment member 504 is a clamp structure, camera 116 may be held in a variety of positions by the clamping members, so that the direction in which camera 116 is pointing may be adjusted as needed. In one embodiment, the clamp structure may allow adjustment of the camera orientation relative to the blade of the laryngoscope. For example, second attachment member 504 may be a clamp structure that is attached to adapter 500 using a ball and socket joint, which allows the clamp structure and attached camera to swivel and/or rotate with respect to the body of adapter 500. Where second attachment member 504 is a receptacle within the adapter body, shims, sleeves, set screws, or other structures may be used to adjust the orientation of camera 116 within the receptacle.

Similarly, adapter 500 may also include a means for adjusting the orientation of the laser with respect to the laryngoscope. For example, the laser orientation may be adjusted such that the laser illuminates the target area proximate to the second end of the laryngoscope blade. Example means for adjusting the orientation of the laser with respect to the laryngoscope may include the clamps, ball joints, swivel joints, shims, sleeves, set screws, and so on, as described above for adjusting the orientation of the camera with respect to the laryngoscope.

In one embodiment, an optically transparent window or lens may be disposed within aperture 508 to protect the laser from contact with and potential contamination by bodily secretions. The window or lens may provide additional conditioning of the laser beam. For example, the window or lens may shape the laser beam so that the laser beam is projected in an arrow or cross-hair shape on the structures being probed. In one embodiment, the window or lens that projects the laser beam in an arrow shape may be rotated within aperture 508 so as to change the direction in which the arrow is pointing. For example, the lens may be disposed within a ring having a knurled outer surface that protrudes from adapter 500. By manually rotating the knurled ring and attached lens, a user may change the orientation of the projected arrow shape.

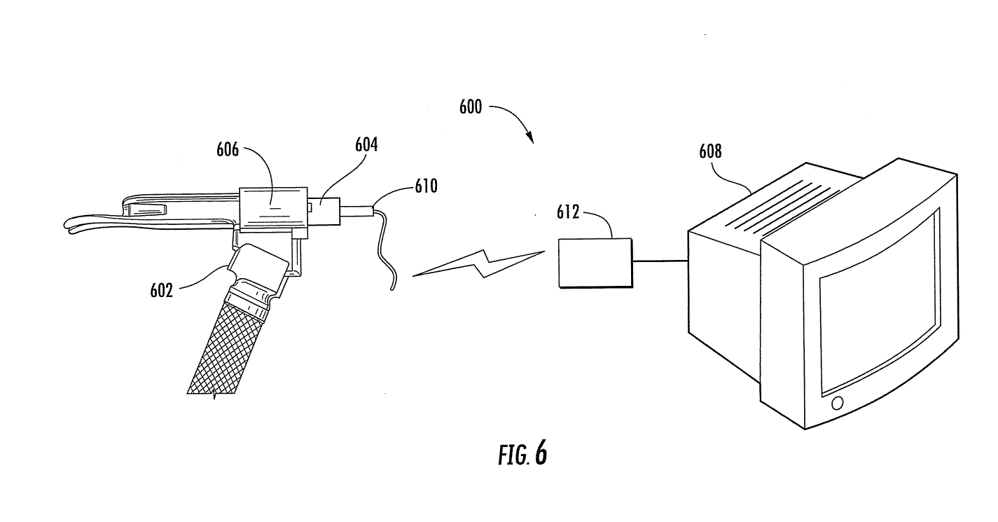

FIG. 6 is an illustration of a system for training laryngoscope users according to an embodiment of the subject matter described herein. System 600 includes a laryngoscope 602, a camera 604 for capturing or collecting images of anatomical structures being probed by laryngoscope 602, an adapter 606 for removably mounting camera 604 to laryngoscope 602 and for holding camera 604 in a predetermined orientation with respect to laryngoscope 602. System 600 also includes a monitor 608 for displaying the output of camera 604. System 600 may include a wireless transmitter 610 for wirelessly transmitting the output of camera 604 to a wireless receiver 612 that is coupled to monitor 608. Camera 604 may output still images or moving images to monitor 608. Camera 604 may capture sound, which may also be played by monitor 608 speakers or other speakers. The audio/visual output of camera 604 may be recorded, such as for later review. In one embodiment, adapter 606 may include an attachment member, such as a receptacle disposed within the adapter body, for holding one or more batteries or battery packs for providing power to devices attached to the laryngoscope, such as camera 604, wireless transmitter 610, a laser, and so on.

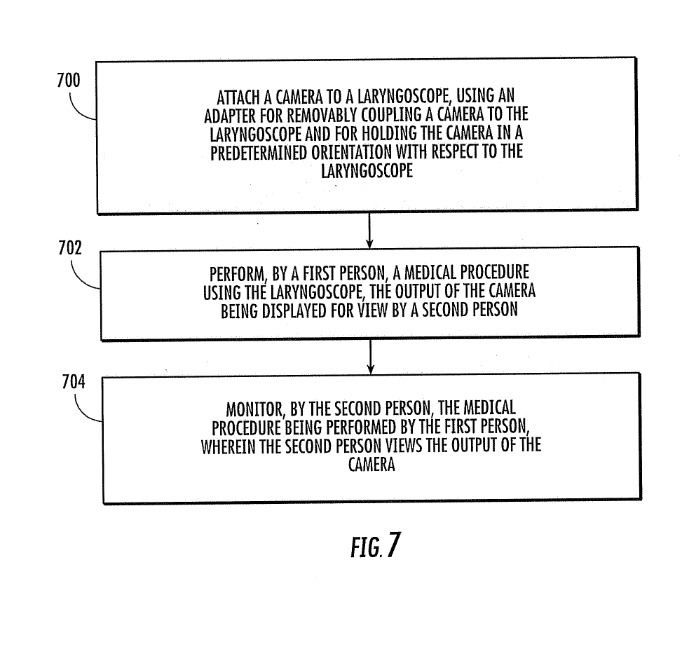

FIG. 7 is a flowchart illustrating an exemplary process for training laryngoscope users according to an embodiment of the subject matter described herein. At block 700, a camera 604 is attached to a laryngoscope 602, using an adapter 606 for removably attaching camera to the laryngoscope and for holding the camera in a predetermined orientation with respect to the laryngoscope. At block 702, a first person performs a medical procedure using laryngoscope 602. The output of camera 604 is displayed on monitor 608. While the first person is using the laryngoscope, a second person monitors the medical procedure being performed by the first person by viewing the output of camera 604 as displayed on monitor 608. In this manner, the second person sees substantially the same view seen by the first person who is performing the medical procedure. For example, the first person may be a trainee who is being trained in the use of the laryngoscope while the second person is the trainer, providing instruction and feedback to the trainee based on the displayed output of the camera. Alternatively, the first person may be a trainer who is showing one or more trainees the view that they will see or should see when they use the laryngoscope. In this scenario, the trainer may use the laser pointer to point out and identify various structural details of the patient's throat, for example.

In an alternative embodiment, the output of the camera may also be viewed by the person using the laryngoscope. For example, in addition to sending the camera output to the monitor, a video splitter may be used to send the camera output to a heads-up display device, such as the laser retinal display device by Microvision, worn by the person using the laryngoscope. The display could be fitted to one lens of eyewear worn by an intubator. For example, the display could be fitted to the left lens, since the left eye is usually blocked by the operator's hand or the laryngoscope blade while the right eye tends to have the clearest unobstructed view of the airway during intubation.

In one embodiment, the camera output could be sent to a remote viewer over wired or wireless connections. The link to the remote viewer may also be a two-way link, allowing the remote viewer to send audio instructions, to remotely control the laser pointer in order to identify structures being remotely viewed, or provide other information back to the intubator, for example. This capability could be very valuable to emergency personnel in the field.

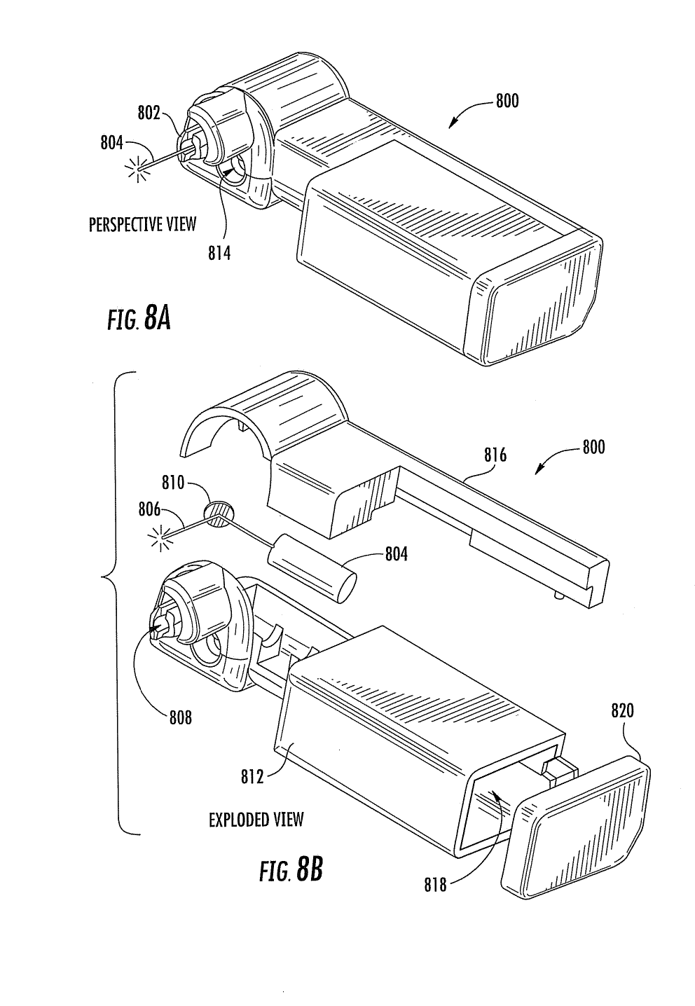

FIGS. 8A and 8B are illustrations of an adaptor for removably coupling a wireless camera to a laryngoscope according to another embodiment of the subject matter described herein. FIGS. 8A and 8B are perspective and exploded views, respectively, of adaptor 800. Adapter 800 includes a structure 802 that functions both as a first attachment member for attaching the body of adapter 800 to a laryngoscope and as a third attachment member for attaching a laser to adapter 800 and for holding the laser in a predetermined orientation with respect to the laryngoscope.

In the embodiment illustrated in FIGS. 8A and 8B, structure 802 forms one wall of a receptacle within the body of adapter 800 for receiving one end of a laryngoscope blade, the other wall of the receptacle being a portion of adapter 800 proximate to structure 802 positioned so that the inserted laryngoscope blade is held between the two walls just described.

In the embodiment illustrated in FIGS. 8A and 8B, structure 802 includes a receptacle disposed within structure 802 for receiving a laser 804. The laser beam 806 is emitted from an orifice or aperture 808 within structure 802. Laser beam 806 is oriented so that it illuminates structures being probed by the user of the laryngoscope, e.g., structures in the patient's throat. In one embodiment, the laser may be a Class 1 laser as specified by the International Electromechanical Commission (IEC) 60825-1 standard (2007), which emits a Class 1 (eye safe) laser beam. In one embodiment, adapter 800 may include a mirror 810, prism, or other means to direct laser beam 806 in the desired orientation or to adjust the orientation of laser beam 806 for the purpose of identifying items within the field of view of the user. For example, the orientation of laser beam 806 may be adjusted or steered, allowing the laser to be used as a laser pointer to facilitate identification of airway objects or orientation of oral structures for subsequent training by an instructor.

In the embodiment illustrated in FIGS. 8A and 8B, adapter 800 includes a bottom portion of adapter body 812 having a second attachment member for attaching a camera to adapter 800, the second attachment member being a receptacle disposed within bottom portion of adapter body 812 for receiving a for receiving a camera. The camera is oriented so as to receive images through orifice or aperture 814. In one embodiment, the camera is a wireless camera, meaning a camera that captures still images, movie images, and optionally sound, and transmits the captured images or sound wirelessly to another device that is not physically electrically connected to the camera. Wireless cameras may transmit captured data by using, for example, radio frequency electromagnetic signals, such as near-field communications, Wi-Fi, RF, Bluetooth, or cellular network; by using light, such as infrared light; or by using another wireless technology suitable for transmitting data wirelessly. The data may be transmitted in encrypted or non-encrypted form, and may be transmitted over an encrypted or non-encrypted wireless network. The data may be wirelessly transmitted to a wireless receiver attached to a personal computer, video phone, video heads-up display, projector, or other video output device.

In one embodiment, a wireless voice over Internet protocol (VoIP) device may be used for voice communication between the user of the laryngoscope and another person located remotely from the user. For example, the VoIP device may be used to facilitate communication between the user of the laryngoscope and a remote instructor during the intubation process. In this manner, the remote instructor can provide real-time training and advice to the user of the laryngoscope.

In the embodiment illustrated in FIGS. 8A and 8B, adapter 800 includes a top portion of adapter body 816, which is removable to gain access to the camera or laser 804. Bottom portion of adapter body 812 includes a receptacle 818 with a removable receptacle cover 820. Receptacle 818 may contain electronics, such as wireless transceivers, memories, batteries, etc. Receptacle 818 and receptacle cover 820 may be designed for access to a battery compartment to allow a user to change batteries or battery packs. For example, receptacle 818 may be designed to receive a nine-volt battery, one or more M or MA cells, or other types of batteries.

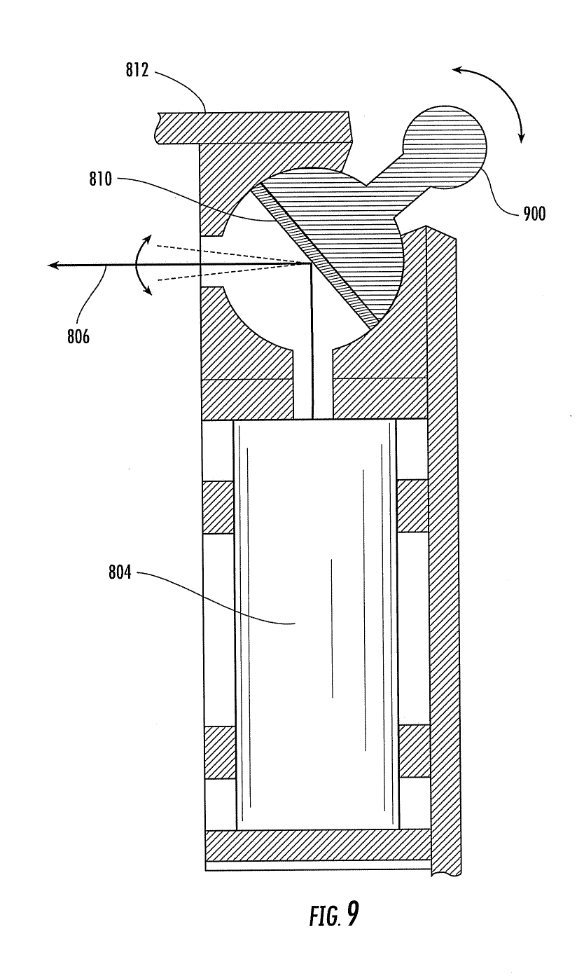

FIG. 9 is a detailed view of the adapter for removably coupling a wireless camera to a laryngoscope illustrated in FIGS. 8A and 8B, illustrating a mechanism for adjusting the orientation of the a laser beam with respect to the the laryngoscope according to another embodiment of the subject matter described herein. FIG. 9 shows laser 804 positioned within the receptacle for that purpose disposed within bottom portion of adapter body 812. Laser 804 emits laser beam 806 into mirror 810, which is oriented so as to reflect laser beam 806 toward the far end of the laryngoscope blade. In the embodiment illustrated in FIG. 9, mirror 810 is attached to structure 900, which can rotate within a circular cavity disposed within bottom portion of adapter body 812 so as to change the angle of mirror 810 relative to the laser beam emitted from laser 804. By changing the angle of mirror 810, the angle at which laser beam 806 exits the circular cavity may be adjusted up and down, thus directing laser beam 806 to illuminate a desired portion of the structures being probed by the laryngoscope user. Although structure 900 appears circular in 2D cross-section in FIG. 9, it will be understood that structure 900 may be half-sphere rotating within a spherical cavity, allowing the angle at which laser beam 806 exits the spherical cavity to be adjusted not only up and down but also from side to side. The structure in FIG. 9 is illustrative and is not intended to be limiting. Other structures which change the orientation of the exiting laser beam 806 are also contemplated.

In an alternative embodiment, the angle of mirror 810 relative to the laser beam emitted from laser 804 may be controlled by electromechanical servos. For example, one servo may control the pitch of the mirror while another servo may control the yaw of the mirror. In this embodiment, the servos may be controlled by controls disposed on adapter 800, such as arrow buttons or a joystick. The controls may have a wired or wireless connection to the servos. In one embodiment, the servos may be manipulated by a remote user. For example, a novice user may receive instructions from a remote instructor, in which case the remote instructor may use the servos to control the position of the laser pointer in order to assist the novice user, such as to point out structures, give directional instructions, and so on.

In one embodiment, the wireless camera may connect via a Bluetooth connection or other wireless connection to a cellular phone or other wireless mobile communication device, such as a wireless VoIP device. The cellular phone may transmit video data captured by the wireless camera to a remote location, along with the audio stream associated with the call. In this manner, a remote user may view the video data being captured by the wireless camera and simultaneously communicate with the user of the laryngoscope as with a normal telephone call over a mobile phone. In such an embodiment, additional equipment, such as a personal computer or laptop and video monitor, would not be necessary, allowing the user of the laryngoscope to be extremely mobile, a desirable benefit for emergency service personnel, for example.

It will be understood that various details of the presently disclosed subject matter may be changed without departing from the scope of the presently disclosed subject matter. Furthermore, the foregoing description is for the purpose of illustration only, and not for the purpose of limitation.

1. An adapter for removably coupling a camera to a laryngoscope, the adapter comprising:
an adapter body, the adapter body including a first attachment member for attaching the adapter body to a laryngoscope having a blade with a first end and a second end; and
the adapter body including a second attachment member for attaching a camera to the adapter body and for holding the camera in a predetermined orientation with respect to the laryngoscope.

2. The adapter of claim 1 wherein the first attachment member comprises a receptacle, disposed within the adapter body, for receiving the first end of the laryngoscope blade and for securing the adapter body to the first end of the laryngoscope blade.

3. The adapter of claim 1 wherein the first attachment member comprises a clamp for clamping the adapter body to the laryngoscope blade.

4. The adapter of claim 3 wherein the clamp is configured to clamp the adapter body to the first end of the laryngoscope blade.

5. The adapter of claim 1 wherein the second attachment member comprises a receptacle, disposed within the adapter body, for receiving the camera.

6. The adapter of claim 5 comprising a retaining clip, disposed within the receptacle, for securing the camera to the adapter body.

7. The adapter of claim 1 wherein the second attachment member comprises a clamp for clamping the camera to the adapter body.

8. The adapter of claim 1 wherein the second attachment member orients the camera generally towards the second end of the laryngoscope blade.

9. The adapter of claim 1 comprising means for mechanically engaging a focusing mechanism of the camera.

10. The adapter of claim 9 wherein the means for mechanically engaging a focusing mechanism of the camera comprises means for rotating a focusing ring of a lens of the camera.

11. The adapter of claim 1 comprising means for adjusting an orientation of the camera with respect to the laryngoscope.

12. The adapter of claim 1, comprising a third attachment member for attaching a laser to the adapter body and for holding the laser in a predetermined orientation with respect to the laryngoscope.

13. The adapter of claim 12 wherein the third attachment member comprises a third receptacle, disposed within the adapter body, for receiving the laser.

14. The adapter of claim 12 wherein the third attachment member comprises a clamp for clamping the laser to the adapter body.

15. The adapter of claim 12 comprising means for adjusting the orientation of the laser with respect to the laryngoscope.

16. The adapter of claim 12 comprising means for adjusting the orientation of a laser beam emitted by the laser with respect to the laryngoscope.

17. The adapter of claim 16 wherein means for adjusting the orientation of the laser beam comprises at least one of a mirror, an optical prism, and a fiber optic conduit.

18. The adapter of claim 1 comprising a wireless transmitter for wirelessly transmitting output of the camera to a wireless receiver, wherein the output of the camera includes at least one of still images, moving images, and sound.

19. A laryngoscope, comprising:
a handle;
a blade having a first end coupled to the handle and a second end adapted to be inserted into the throat of a patient;
a camera for imaging anatomical structures being probed by the laryngoscope; and
an adapter for removably coupling the camera to the laryngoscope blade and for holding the camera in a predetermined orientation with respect to the laryngoscope blade.

20. The laryngoscope of claim 19 wherein the adapter comprises an adapter body, the adapter body including a first attachment member for attaching the adapter body to the laryngoscope blade and a second attachment member for attaching the camera to the adapter body and for holding the camera in the predetermined orientation with respect to the laryngoscope blade.

21. The laryngoscope of claim 20 wherein the first attachment member comprises a first receptacle, disposed within the adapter body, for receiving the first end of the laryngoscope blade and for securing the adapter body to the first end of the laryngoscope blade.

22. The laryngoscope of claim 20 wherein the first attachment member comprises a clamp for clamping the adapter body to the laryngoscope blade.

23. The laryngoscope of claim 22 wherein the clamp is configured to clamp the adapter body to the first end of the laryngoscope blade.

24. The laryngoscope of claim 20 wherein the second attachment member comprises a second receptacle, disposed within the adapter body, for receiving the camera.

25. The laryngoscope of claim 24 comprising a retaining clip, disposed within the second receptacle, for securing the camera to the adapter body.

26. The laryngoscope of claim 20 wherein the second attachment member comprises a clamp for clamping the camera to the adapter body.

27. The laryngoscope of claim 20 wherein the camera is oriented to collect images in a direction of the laryngoscope blade.

28. The laryngoscope of claim 20 comprising means for mechanically engaging a focusing mechanism of the camera.

29. The laryngoscope of claim 28 wherein the means for mechanically engaging a focusing mechanism of the camera comprises means for rotating a focusing ring of a lens of the camera.

30. The laryngoscope of claim 20 comprising means for adjusting the orientation of the camera with respect to the blade.

31. The laryngoscope of claim 20, comprising:
a laser; and
a third attachment member for attaching the laser to the adapter body and for holding the laser in a predetermined orientation with respect to the laryngoscope.

32. The laryngoscope of claim 31 wherein the third attachment member comprises a receptacle, disposed within the adapter body, for receiving the laser.

33. The laryngoscope of claim 31 wherein the third attachment member comprises a clamp for clamping the laser to the adapter body.

34. The laryngoscope of claim 31 comprising means for adjusting the orientation of the laser with respect to the blade.

35. The laryngoscope of claim 31 comprising means for adjusting the orientation of a laser beam emitted by the laser with respect to the blade.

36. The laryngoscope of claim 35 wherein means for adjusting the orientation of the laser beam comprises at least one of a mirror, an optical prism, and a fiber optic conduit.

37. The laryngoscope of claim 31 wherein the laser comprises a laser that emits an eye-safe laser beam.

38. The laryngoscope of claim 37 wherein the laser is a Class 1 laser.

39. The laryngoscope of claim 20 comprising a wireless transmitter for wirelessly transmitting output of the camera to a wireless receiver, wherein the output of the camera includes at least one of still images, moving images, and sound.

40. A system for training laryngoscope users, the system comprising:
a laryngoscope having a blade;
a camera for imaging anatomical structures being probed by the laryngoscope;
an adapter for removably coupling a camera to the laryngoscope and for holding the camera in a predetermined orientation with respect to the laryngoscope; and
a monitor for displaying output of the camera.

41. The system of claim 40 wherein the monitor is oriented to allow a person who is not using the laryngoscope to see substantially the same view as the view seen by a person who is using the laryngoscope.

42. The system of claim 40 wherein the adapter comprises an adapter body, the adapter body including a first attachment member for attaching the adapter body to the laryngoscope blade and a second attachment member for attaching the camera to the adapter body and for holding the camera in the predetermined orientation with respect to the laryngoscope blade.

43. The system of claim 42 wherein the first attachment member comprises a receptacle, disposed within the adapter body, for receiving a first end of the laryngoscope blade and for securing the adapter body to the first end of the laryngoscope blade.

44. The system of claim 42 wherein the first attachment member comprises a clamp for clamping the adapter body to the laryngoscope blade.

45. The system of claim 44 wherein the clamp is configured to clamp the adapter body to a first end of the laryngoscope blade.

46. The system of claim 42 wherein the second attachment member comprises a receptacle, disposed within the adapter body, for receiving the camera.

47. The system of claim 46 comprising a retaining clip, disposed within the receptacle, for securing the camera to the adapter body.

48. The system of claim 42 wherein the second attachment member comprises a clamp for clamping the camera to the adapter body.

49. The system of claim 40 wherein the camera is oriented to collect images in a direction of the laryngoscope blade.

50. The system of claim 40 comprising means for mechanically engaging a focusing mechanism of the camera.

51. The system of claim 50 wherein the means for mechanically engaging a focusing mechanism of the camera comprises means for rotating a focusing ring of a lens of the camera.

52. The system of claim 40 comprising means for adjusting the orientation of the camera with respect to the blade.

53. The system of claim 40 wherein the adapter comprises an adapter body, and wherein the system further comprises a laser and a third attachment member for attaching the laser to the adapter body and for holding the laser in a predetermined orientation with respect to the laryngoscope.

54. The system of claim 53 wherein the third attachment member comprises a receptacle, disposed within the adapter body, for receiving the laser.

55. The system of claim 53 wherein the third attachment member comprises a clamp for clamping the laser to the adapter body.

56. The system of claim 53 comprising means for adjusting the orientation of the laser with respect to the blade.

57. The system of claim 53 comprising means for adjusting the orientation of a laser beam emitted by the laser with respect to the blade.

58. The system of claim 57 wherein means for adjusting the orientation of the laser beam comprises at least one of a mirror, an optical prism, and a fiber optic conduit.

59. The system of claim 53 wherein the laser comprises a laser that emits an eye-safe laser beam.

60. The system of claim 59 wherein the laser is a Class 1 laser.

61. The system of claim 40 comprising a wireless transmitter for wirelessly transmitting output of the camera to a wireless receiver, wherein the output of the camera includes at least one of still images, moving images, and sound.

62. A method for training laryngoscope users, the method comprising:
attaching a camera to a laryngoscope, using an adapter for removably coupling a camera to the laryngoscope and for holding the camera in a predetermined orientation with respect to the laryngoscope;
performing, by a first person, a medical procedure using the laryngoscope, the output of the camera being displayed for view by a second person; and
monitoring, by the second person, the medical procedure being performed by the first person, wherein the second person views the output of the camera.
